(19) United States
(12) Patent Application Publication
(10) Pub. No.: US 2004/0116369 A1
(43) Pub. Date: Jun. 17, 2004

(54) Interferon regulatory factor-1/human estrogen receptor fusion protein and its use for treating carcinomas

(76) Inventors: Andrea Kroeger, Braunschweig (DE); Michael Geissler, Braunschweig (DE); Hansjoerg Hauser, Braunschweig (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/648,454

(22) Filed: Aug. 26, 2003

Related U.S. Application Data
(63) Continuation-in-part of application No. PCT/EP02/02036, filed on Feb. 26, 2002.

(30) Foreign Application Priority Data
Feb. 26, 2001 (EP) 01104423.7

(51) Int. Cl. A61K 48/00

(52) U.S. Cl. 514/44

(57) ABSTRACT

The invention relates to an interferon regulatory factor-1/human estrogen receptor fusion protein which is reversibly activatible by β-estradiol and its use for treating carcinomas, particularly for treating hepatocellular carcinoma.

This is a continuation-in-part of International Application No. PCT/EP02/02036 filed Feb. 26, 2002, the entire disclosure of which is incorporated herein by reference.

### BACKGROUND OF THE INVENTION

### FIELD OF THE INVENTION

The invention relates to the development of methods for treating tumors. The methods are based on IRF 1, particularly the activation of an IRF 1/human estrogen receptor fusion protein which is reversible activatable by β-estradiol.

### ABBREVIATIONS

The following abbreviations are used in this description:

AFP=alpha fetoprotein

c-Ha-ras=ras-oncogen

c-myc=myc oncogen

CTL=cytotoxic T lymphocyte

E2=β-estradiol

FCS=fetal calf serum

fosB=transcription factor fosB

HCC=hepatocellular carcinoma cell

HER1=EGF receptor

ICE=caspase

IL-15=interleucin-15

iNOS=inducible NO synthase

ISG=interferon stimulated gene

ISGF3=IRF-1 related subunit

ISRE=interferone stimulated response element

LMP2=Protein processing factor

mAFP=murine alpha fetoprotein

MECL1=multicatalytic endopeptidase complex 1

MHC=Major histocompatibility complex

NK cell=natural killer cell

OASE=2′,5′-Oligo (A) Synthetase

PKR=Protein Kinase R

ras induction=induction of the ras protein

TAP-1=Protein processing factor

TH1=T helper cell type 1

A complete list of the cited references with detailed bibliographic information can be found at the end of the description.

### DESCRIPTION OF RELATED TECHNOLOGY

Hepatocellular carcinoma (HCC) ranks fifth in frequency among all malignancies in the world with an estimated number of 437,000 new cases in 1990 (1). Although various non-surgical treatment modalities have been developed and the surgical techniques much improved, none of these therapies has significantly improved the extremely poor prognosis of patients with HCC. The overall 5-year survival rate worldwide is only 2% (2) and, therefore, novel gene and immunotherapeutic strategies for HCC are being developed. The inventors attempted to employ the broad role of interferon regulatory factor-1 (IRF-1) (3) as a tumor suppressor and immune modulator for the treatment of tumors. They used an immunocompetent syngeneic HCC tumor model in mice and another tumor cell line for test in immunodeficient mice.

IRF 1 expression leads to the induction of many interferon stimulated genes (ISGs) (4-6) and thereby induces typical IFN functions including induction of histocompatibility antigens (7) and an antiviral state (5, 8). Since the IRF-1 gene per se is inducible by IFNs it was suggested that it might be involved in IFN mediated cellular responses (5, 9, 10). However, in mice and cells lacking functional IRF-1 genes the IFN induced induction of typical ISGs (e.g., 9-27, 1-8, PKR) is not affected (8, 11-13). Thus, IRF-1 seems to stimulate the IFN-specific induction of ISGs by ISGF-3 which binds the ISG promoter with the IRF-1 related subunit ISGF3 (14). A specific alteration in these mice is the lack of iNOS (inducible NO synthase) induction in response to IFN-β (15).

IRF-1 exerts an antiproliferative effect by DNA binding and transactivation (4). It is known to induce a number of genes which exert growth inhibitory effects. Among them are Lysyl oxidase (16), PKR (17), 2′-5′ OASE (18), Indoleamine 2,3-dioxigenase (19), and Angiotensin type II receptor (20). In established cell lines of fibroblast and epithelial origin, IRF-1 leads to cell growth arrest without signs of apoptosis (21). However, similar to the activity of the tumor suppressor p53 required for ras induced apoptosis (22, 23), IRF-1 is able to exert oncogene dependent apoptosis. The inventors have shown that 3T3 cells which are growth inhibited by IRF-1, undergo apoptosis after conditional HER1 oncogene activation (24). Indeed, the promoter regions of certain caspase genes like ICE contain ISRE-like sequences (25). These genes might be targets for IRF-1 (26).

IRF-1 has been identified as a tumor suppressor (4, 17, 24, 27). Chromosomal deletions of the IRF-1 locus in humans are associated with myelodysplasia and certain leukemias (28). Primary embryonic fibroblasts with a null mutation in the IRF-1 gene are susceptible to transformation by the expression of a single oncogene (c-Ha-ras). These IRF 1^{-/-} cells do not undergo apoptosis upon c-Ha-ras oncogene expression and serum starvation while wild type cells harboring IRF-1 genes undergo programmed cell death (21). IRF-1 expression also reverts the tumorigenic phenotype exerted by the c-myc and fosB oncogenes (29). Further data indicated that mice lacking c-Ha-ras and IRF-1 exhibit a higher rate of tumorigenicity (30).

The in vivo function of IRF-1 as a tumor suppressor is complex. Depending on the cell type, IRF-1 induces growth inhibition, apoptosis, effects the extracellular matrix as well as immunomodulatory functions. IRF-1 induces a number of immunomodulatory effects like MHC class I (31, 32), iNOS (15), IFN-β (11) transcription, and is also necessary for proper expression of IL-15 (33). Transient expression of IRF-1 leads to the activation of the IFN β gene (9, 34, 35). Studies with IRF-1 knock-out cells demonstrate that IRF-1 is involved in the differentiation and function of NK cells (33, 36), in the generation of the TH1 type of T helper cells, and DNA damage (26, 37). IRF-1 is further involved in up-regulation of the antigen presentation by transcriptional induction of LMP2, TAP-1 and MECL1 (38, 39) as well as an induction of MHC class II (7, 40). These facts suggest that IRF-1 might act as a costimulator for presentation of antigens.

### SUMMARY OF THE INVENTION

It is an objective of the invention to provide the therapeutic approaches for tumors using IRF-1. The invention provides construction of plasmids and murine cell lines encoding an IRF-1/human estrogen receptor fusion protein, which becomes active in the presence of β-estradiol (E2) (4) and the detailed characterization of the in vitro phenotype of these cell lines. Furthermore, protective and therapeutic potential of this activatable IRF-1 system against tumor growth in vivo using immune competent and incompetent mice and the characterization of T cell response against the tumor are described, and it is demonstrated that immunologic tolerance to the HCC specific self differentiation antigen mouse a-fetoprotein (AFP) (41) can be broken by this approach. It is also demonstrated that IRF-1 mediates its antitumoral effects through both a direct antitumor growth effect and through enhanced immune cell recognition of the tumor.

### DETAILED DESCRIPTION OF THE INVENTION

The problem of the invention can be solved by the use of a gene construct or a polynucleotide by means of which the activity of its gene product, a member of the IRF family, can become activated, for the treatment, prevention, protective treatment and/or prophylactic immunization against tumoral, infectious and/or immune diseases. A characteristic embodiment of the invention comprises the use as mentioned above, wherein the member of the IRF family as gene product is selected from the group consisting of wild-type IRF-1, synthetic IRF-1, immunologically active IRF-1 variants, a wild-type member of the IRF family other than IRF-1, a synthetic member of the IRF family other than IRF-1, immunologically active variants of a member of the IRF family other than IRF-1, and fusion proteins thereof. The invention relates to the use as mentioned above for the treatment of mammals and especially humans.

A further embodiment comprises the use as mentioned above, wherein the gene construct encodes a member of the IRF family as a fusion protein comprising said member as one of the domains of the fusion protein and a foreign protein as another domain of the fusion protein, wherein the activity of the fusion protein can be switched on and off by chemical or physical means, especially, wherein the gene construct encodes the expression of an IRF-1/hER fusion protein, the activity of which can be regulated by compounds with estrogenic or anti-estrogenic activity.

A further embodiment comprises the use as mentioned above, wherein the gene construct or the polynucleotide is provided as a product for transfer into mammalian cells, especially a viral vector, preferably an adenoviral vector.

A vaccine with an adenoviral vector comprising an expression construct for the expression of an IRF-1/hER fusion protein is highly efficient in suppressing tumor development and can therefore be used as an active therapeutic and/or prophylactic vaccine. Adenoviruses showing the potential to express an IRF-1/hER fusion protein can be gained in high titers if the IRF-1/hER fusion protein is in its inactive/uninduced state without estradiol. Recombinant adenoviruses are difficult to obtain if the IRF-1/hER fusion protein is activated during the viral production.

A characteristic feature of the invention consists of the expression of proteins mentioned above which can be switched on by chemical or by physical activation, for example thermally or by irradiation. A further characteristic feature consists of the expression of said proteins, wherein the expression can be switched on or off by means of a regulatable promoter, for example by an external stimulus, especially by tetracyclines.

An advantageous embodiment of the invention consists of a vaccine comprising a gene construct or a polynucleotide as defined in one or more of the preceding claims, together with one or more antigens selected from the group consisting of viral, bacterial, fungal, and parasitic origin or one or more antigen encoding genes derived from tumor cells. The advantage of this embodiment consists of avoiding the use of tumor cells. The vaccination can be carried out by applying merely a gene construct or polynucleotide and the antigen encoding sequence(s) are provided by means of separate vectors, a vector which provides all components, or as polycistronic expression units. A further advantageous embodiment comprises a vaccine as mentioned above, wherein the gene construct or polynucleotide and the antigen encoding sequence(s) are provided as viral or bacterial carrier.

Another embodiment of the invention consists of the use as mentioned above for the production of preparations for the treatment, prevention, protective treatment and/or prophylactic immunization of tumoral, infectious and/or immune diseases.

Further, another embodiment relates to a member of the IRF family or a fusion protein comprising or consisting of said member as one of the components of the fusion protein and a therapeutically acceptable protein as another component of the fusion protein for the treatment, prevention, protective treatment and/or prophylactic immunization of tumoral, infectious and/or immune diseases, especially a fusion protein, wherein IRF-1 is one of its components and/or hER is the other component of the fusion protein.

The invention comprises further a prophylactic and/or therapeutic composition consisting of or comprising a member of the IRF family or a fusion protein comprising or consisting of said member as one of the components of the fusion protein and a therapeutically acceptable protein as another component of the fusion protein for the treatment, prevention, protective treatment and/or prophylactic immunization of tumoral, infectious and/or immune diseases, especially a composition, wherein IRF-1 is one of the components of the fusion protein and/or hER is the other one of its components.

Additionally, the invention concerns a human cell charged ex vivo with a gene construct or polynucleotide as mentioned above for the expression of a member of the IRF family, preferably by charging a cell by gene transfer, physical transduction, more preferably by electroporation, chemical transduction or viral transduction. The human cell mentioned above may be an autologous or an allogenic cell. Further, the human cell mentioned above may be a tumor cell, preferably a carcinoma cell, more preferably a hepatocellular carcinoma cell, a sarcoma cell or a tumor derived from the hematopoietic system. Additionally, the human cell mentioned above is suited for the treatment, prevention, protective treatment and/or prophylactic immunization against tumoral, infectious and/or immune diseases.

A preferred advantageous embodiment of the invention relates to a human antigen presenting cell (APC), wherein the cell has been subjected to a gene transfer, physical transduction, especially electroporation, chemical transduction or viral transduction, with one or more genes mentioned in the embodiments of vaccination.

Finally, the invention contains an embodiment of a human cell as mentioned above, wherein the active level of the member of the IRF family is higher than that of IRF-1 induced by interferon-beta.

The objects of the invention, the various features thereof, as well as the invention itself, may be more fully understood from the following description, when read together with the accompanying figures/drawings, in which shows:

### BRIEF DESCRIPTION OF THE DRAWINGS

FIG. 1. IRF-1 mediates cell growth inhibition in Hepa 1-6 cells. A: Western blot analysis of IRF-1hER fusion protein in Hepa1-6 cells transfected with IRF-1hER. Lysates (50 μg of protein) derived from these cell clones were subjected to SDS-PAGE and analyzed using an anti-ER antibody (top panel). The membrane was stripped and reprobed with an anti-actin antibody to control protein loading of the samples (bottom panel). B: For determination of cell growth, cells derived from the same clones were seeded in wells of microtiter plates and grown with (black bars) or without (white bars) 1 μM β-estradiol. The metabolic activity of the cultures was measured after seven days of treatment. Since the growth characteristics of individual Hepa1-6 cell clones differ slightly, the metabolic activity of untreated cells was set as 100%.

FIG. 2. Activation of IRF-1 reverts the tumorigenic phenotyp of Hepa 1-6 cells. A: Anchorage independent growth of IRF-1 transfected cell clones in soft agar. B: The number of colonies of the indicated cell clones was determined after three weeks culture without (black bars) and with (white bars) 1 μM β-estradiol.

FIG. 3. Activation of IRF-1 leads to increased MHC class I and MHC class II expression. FACS analysis of surface proteins of Hepa 1-6 cells expressing IRF-1-hER (clone 9) was done using antibodies directed against H2K^{b}/D^{b} (A), I-A/I-B (B), and CD54 (C). HepaIRF-1hER cells were cultured for three to four days in estrogen free medium or in medium containing a 1:1,000 dilution of 1 mM β-estradiol. Subsequently, cells were stained with the appropriate FITC- or PE-labeled antibodies. As controls for specificity cells were stained with FITC- or PE-labeled unspecific isotype controls. Data are derived from 3 independent experiments.

FIG. 4. Adenoviral transduction of IRF-1-hER leads to IRF-1 mediated phenotypes.

A, Schematic presentation of the different adenoviral vectors as described in the material and method section. Arrow: Cytomegalovirus promoter (CMV); Bidirectional arrow: bidirectional tTA-Promoter (bitTA); Black dots: SV40 polyadenylation signal; black rectangle: polio virus derived internal ribosomal entry site (IRES); eGFP: enhanced green fluorescence protein; LTR: Retroviral long terminal repeat; tTA: transactivator. B, Western Blot analysis of IRF-1-hER fusion protein in Hepa1-6 cells infected with the indicted adenoviruses. Lysates (50 μg of protein) derived from cells 48 h after infection were subjected to SDS-PAGE and analyzed using antibodies directed against the human estrogen receptor. C, Anchorage independent growth of Hepa1-6 cells in soft agar. The upper row shows Hepa1-6 cells in culture without (−E2) and the lower row shows Hepa1-6 cell in culture with (+E2) β-estradiol. In the left column, non-infected (control) Hepa1-6 cells are shown. Cells in the middle and right column were transfected with the indicated adenoviruses. Cells were seeded 48 h after infection.

FIG. 5. Activation of IRF-1 retards and partially inhibits tumor growth in nude mice.

10⁶ cells per mouse were injected subcutaneously into right flank of NMRI nude mice. Mice were either treated with 1.5 mg E2 every two days i.p. or left untreated. Data represent mean values of five animals. A, Tumor growth was measured by the tumor volume. B, Kaplan-Meier plot showing the percentage of tumor free nude mice survival.

FIG. 6. In vivo characteristics of HepaIRF-1hER and Hepa1-6 cells dependent on E2 treatment in syngeneic immunocompetent C57L/J mice and protection against tumor rechallenge. A: Mice were inoculated with 1×10⁷ Hepa1-6 or HepaIRF-1hER cells into the right flank and either treated with 1.5 mg β-estradiol every two days i.p. or left untreated. Note that six out of eight E2 treated animals were completely protected against HepaIRF-1hER tumor growth and two animals developed only small tumors which were characterized by a very slow growth rate even after stopping E2 treatment. B: Naive C57L/J mice or E2 treated animals protected against HepaIRF-1hER tumor growth were rechallenged with 1×10⁷ wild-type Hepa1-6 (n=3) or HepaIRF-1hER (n=3) tumor cells 11 days after stopping E2 treatment (28 days after the initial tumor inoculation). Importantly, these mice did not receive any further E2 treatment.

FIG. 7. CTL activity and T cell precursor frequency against Hepa1-6 tumors. A: Spleen cells derived from tumor challenged or control mice (n=5 in each group) were re-stimulated using irradiated Hepa1-6 cells and subsequently analyzed for cytotoxic activity against syngeneic Hepa1-6 and Lewis lung carcinoma cells at the E:T ratios indicated. For control of specificity, E2 treated mice challenged with HepaIRF-1hER cells were in vitro stimulated with 3LL cells and subsequently analyzed for cytotoxic activity against syngeneic Hepa1-6 and 3LL carcinoma cells. Hepa1-6 tumor specific lysis was presented by subtraction of lysis values against 3LL from lysis values against Hepa1-6 targets. Values represent means of triplicate determinations. B: Spleen cells derived from tumor challenged or unchallenged mice (n=5 in each group) were stimulated for 20 hours with Hepa1-6 or 3LL cells. Subsequently, IFN-γ and IL-4 ELISPOT assays were performed. The spots in each well were counted under a microscope, and the values are expressed as numbers of spot-forming cells relative to the number spleen cells added to each well at the start of the culture.

FIG. 8. AFP specific CTL responses and CTL-p frequencies. A: Spleen cells derived from tumor challenged or control mice (n=5 in each group) were restimulated using irradiated autologous spleen cells infected with UV-inactivated rVV-mAFP and subsequently analyzed for cytotoxic activity against syngeneic Hepa1-6 and Lewis lung carcinoma cells at the E:T ratios indicated. For control of specificity, E2 treated mice challenged with HepaIRF-1hER cells were in vitro stimulated with spleen cells infected with rVV-pSC11 and subsequently analyzed for cytotoxic activity against syngeneic Hepa1-6 and Lewis lung carcinoma cells. AFP specific lysis was presented by subtraction of lysis values against 3LL from lysis values against Hepa1-6 targets. Values represent means of triplicate determinations. B: Spleen cells derived from tumor challenged or unchallenged mice (n=5 in each group) were stimulated for 20 hours with UV-inactivated rVV-AFP or rVV-pSC11 infected syngeneic irradiated spleen cells. Subsequently, IFN-γ ELISPOT assays were performed.

FIG. 9. Identification of antitumoral immune reactivities in vivo and IRF-1 mediated HCC therapy. A: Antitumoral immunity partially required the participation of both CD4+ and CD8+ T cells. CD4 and CD8 T cell subpopulations of E2 treated or untreated mice inoculated with HepaIRF-hER cells were depleted by i.p. injection of purified hybridoma supernatant as described in Materials and Methods. Each group contained two mice. B: C57L/J mice were inoculated with 1×10⁷ HepaIRF-1hER (groups 1 and 2) cells into the right flank. Both groups did not receive any E2 treatment. At day 19 tumors had reached a size of about 2000 mm³ in both groups. Starting at day 19 group 2 (n=6) was i.p. injected with 1.5 mg E2 every two days until day 28. Subsequently, E2 treatment was stopped again in two mice whereas the remaining four mice were maintained on E2 therapy. No differences in tumor growth was observed. Mice of group 1 (n=3) did not receive E2 treatment and were subsequently sacrificed if tumors had reached 10,000 mm³.

In the following the invention is disclosed in more detail with reference to examples and to drawings. However, the described specific forms or preferred embodiments are to be considered in all respects as illustrative and not restrictive, the scope of the invention being indicated by the appended claims rather than by the following description, and all changes which come within the meaning and range of equivalency of the claims are therefore intended to be embraced therein.

### Materials and Methods

Vector Construction:

pBTTAHis: The BbrPI/NotI fragment containing the hisdidinol gene was isolated from pDAF2HIS (Spitzer et al., 1998) and inserted into the correspondingly restricted pRBTtTA (Unsinger et al., 2001). The resulting plasmid is entitled pBTTAHis. For stably transfection of Hepa 1-6 cells we used an expression construct IRF-1-hER (pMT7RF-1-hER) (4) and a puromycin resistence conferring plasmid (42). pHBTMRS: The EcoRI/NotI fragment containing the c-myc, the c-Ha-ras and the SEAP gene as a tricistronic expression cassette was inserted on the 3′ end of the bidirectional tetracycline responsible promoter from pRBT (Unsinger et al., 2001). Hyromycin-B-phospotransferase was PCR amplified and inserted via PmeI 5′ from the bidirectional tetracycline responsible promoter in pRBTMRS resulting in pHBTMRS (Kröger, 1999). pCMVIHEG: The SpeI/EcoRI fragment containing the IRF-1hER fusion protein gene and a polio IRES sequence were inserted between a CMV promoter and the eGFP gene resulting in a plasmid expressing IRF-1hER and eGFP on a bicistronic mRNA. pCMVIH: The sequence encoding for polio IRES and eGFP was eliminated by digestion and religation of pCMVIHEG with PmaCI/HpaI. pLTR-TBTIHEG: The PmeI/NotI fragment containing the bicistronic expression cassette for IRF-1-hER and eGFP were inserted into the correspondingly restricted LTR-THTG (Unsinger et al., 2002) resulting in pLTR-TBTIHEG.

Preparation of Adenovirus Cosmids:

Ad-IH, Ad-IHEG, Ad-IHEGinv, Ad-LTR-TBTIHEG, Ad-LTR-TBTIHEGinv: Recombinant adenoviruses were constructed using a cosmid cloning procedure which allows direct assembly of recombinant adenovirus genomes by cloning in E. coli. Cosmid vector pAdcos45 (Unsinger et al., 2002) was digested with XbaI at a single cloning site in the E1 region and filled in. The following cassettes were inserted into this site: the CMVIHEG cassette of pCMVIHEG as a PmeI/SwaI fragment, the LTR-TBTIHEG of pLTR-TBTIHEG as BsrBI/FspI fragment and the CMVIH cassette from pCMVIH as Pme/SwaI fragment. DNA was ligated and packaged in vitro using packaging extracts. Ampicillin resistant clones were isolated after tranduction into E. coli DH5α. Cosmid praparations for Ad-IH, Ad-IHEG, Ad-IHEGinv, Ad-LTR-TBTIHEG, Ad-LTR-TBTIHEGinv were obtained after in vitro packaging and propagation in E. coli. Restriction analysis confirmed the expected structures.

Cell Culture and Gene Transduction:

Murine fibroblast NIH3T3 cells (ATCC CRL-1658) were maintained in Dulbecco's modified Eagle's medium (DMEM), Hepa1-6 cells (murine H-2b-restricted HCC cell line; 92110305; European Collection of Animal Cell Cultures) were grown in RPMI 1640 medium and 293 cells (human embryonal kidney cells at low passages; Microbix PD-02-01) were cultivated in minimum essential medium (MEM). Media were supplemented with 10% estrogen-free, heat inactivated fetal calf serum, 2 mM L-glutamine penicillin (10 U/ml) and streptomycin (100 μg/ml). Transfected NIH3T3 cells were selected with 128 U/ml hygromycin B, 800 μg/ml histidinol or 800 μg/ml G418. For selection of Hepa1-6 cells 1 μg/ml puromycin was used.

NIH3T3 cells were stably cotransfected with pBTTAHis, pHBTMRS, pMT7IRH-1-hER and the neomycin resistence-conferring plasmid pAG60 (Colbere et al., 1981). Hepa1-6 cells were stably cotransfected using an expression construct encoding IRF-1-hER (4) and and puromycin resistence-conferring plasmid (42). Transfectants were selected and single clones were picked and expanded. Clones were subsequently screened for protein expression by Western blotting.

### PRODUCTION OF RECOMBINANT ADENOVIRUSES

For the production of recombinant adenoviruses 20 μg of circular adenovirus cosmid DNA was transfected into 293 helper cells using calcium phosphate coprecipitation. 10 to 14 d after transfection when the formation of adenoviral plaques became evident. Viruses were harvested and 293 cells were reinfected. After cytopathic effects were observed virus particles were harvested and the titer determined by infection of 293 cells. DNA (Graham et al., 1991) confirmed the expected structures, that is correct excision of the viral sequences from the cosmid DNA was controlled by restriction analysis. For adenoviral infection cells were seeded in MEM medium. The following day cells were infected using an MOI of 100 (Hepa1-6 cells) or 2 (293 cells). Recombinant adenoviruses were diluted in 1 ml PBS supplemented with 2% FCS. One hour after adenoviral infection cells were further cultivated in medium supplemented with 5% FCS.

For activation of IRF-1 in the IRF-1-hER fusion protein, E2 (SERVA, Frankfurt, Germany) was added to the cell culture medium to reach a final concentration of 1 μM.

Western Blotting. Immunoblots derived from whole cell extracts were probed with antibodies directed against the hormone binding domain of the human estrogen receptor (HC-30, Santa Crutz Biotechnology, USA) and visualized by ECL (Amersham, Arlington Heights, Ill., USA) according to manufacturer's specifications.

IFN-Test. The interferon concentrations in the cell culture supernatants were determined by an antiviral assay imploying mouse L929 cells (43). To confirm the specificity of the antiviral activity a neutralizing monoclonal antibody directed against mouse IFN-β was added to the supernatant before addition to the test cells.

Measurement of cell growth. For determination of cell growth 2×10³ cells/well were seeded into microtiter plates and serial dilutions (1:1) were performed allowing several independent measurement points. Cells were treated with the indicated concentration of β-estradiol. Cell growth was determined using the WST kit (Roche Diagnostics, Mannheim, Germany) following the manufacturer's instruction. Mean values of triplicates resulting in less than 10% deviation were plotted.

Assay for anchorage independent growth. Anchorage-independent growth capability was determined by assessing the colony-formation efficiency of cells suspended in soft agar. 1×10³ cells were seeded in 50 μl of 0.3% overlay agar in microtiter plates coated with 50 μl 0.6% underlay agar. The induction medium was added to the top (50 μl/well). Colonies were counted three weeks after plating.

Mice. Male C57L/J (H-2b) mice were kept in the animal facility of the University Hospital Freiburg and used between the age of 10 to 25 weeks.

Nude Mice Experiments

Male 6-8-week old NMRI nude mice (Harlan Winkelmann, Borchen, Germany) were maintained in the SPF unit of the animal facility of German Research Center for Biotechnology. Mice were divided into two experimental groups, five mice for each group. 1×10⁶ cells in 0.2 ml PBS NIH3T3TA/MR/IH cells were injected subcutaneously into the flanks of the mice. The groups were treated as follows: group 1: no treatment (n=5); group 2: 1.5 mg of E2 every two days i.p. (n=4). Tumor volumes were measured and recorded three times a week using calipers. Data are presented as mean value of tumor volume.

Tumor model. The Hepa1-6 tumor model in C57L/J mice (44) was chosen because they show reliable growth in the syngeneic host. Hepa1-6 cells are a derivative of the BW7756 mouse hepatoma that arose in a C57L mouse. MHC class I and II expression is identical between C57L and C57L/J mice and Hepa1-6 HCCs are characterized by AFP expression. It could be demonstrated that a reliable tumor growth of Hepa1-6 murine HCC cells in 100% of mice was achieved using 1×10⁷ or 5×10⁶ Hepa1-6 cells injected in 100 μl serum free MEM medium into the right flank of mice. After 6 days, tumors were visible and reached a size of about 2000 mm³ after a mean time of 18 days. This tumor size was used as endpoint in the invention, and mice were subsequently sacrificed. Tumor incidence and volume were assessed every two days using calipers. Data are presented as mean volume±SE.

Flow cytometry. MHC class I and CD80 expression in Hepa1-6 and HepaIRF-1hER cells were examined by FACS analysis using an anti-mouse H-2Kb/H-2Db and anti-mouse CD80 specific antibody (clones 28-8-6 and 01940B, respectively) and a subsequent FITC-labeled anti-mouse (clone 02014D) or FITC-labeled anti-rat (clone 10094D) antibody, respectively. Furthermore, expression of CD54 (clone 01544D), I-A/I-E (clone 06355A), and CD86 (clone 09274) was determined by PE- or FITC-labeled antibodies (all antibodies derived from PharMingen, San Diego, Calif., USA).

Generation of recombinant vaccinia viruses. To study CTL responses an AFP expressing and pSc11 (empty vector negative control) recombinant vaccinia virus were generated as previously described (44).

Cytotoxicity Assays. Spleen cells derived from tumor challenged or control mice were suspended and after six days of in vitro stimulation in 24 well plates the spleen cells were analyzed for cytotoxic activity. In vitro stimulation was performed by incubating 4×10⁷ of tumor primed spleen cells with 1×10⁷ wild-type Hepa1-6 cells or, as a negative control for specificity, syngeneic Lewis lung carcinoma cells (3LL), both irradiated with 8000 rad. To assess AFP specific immune responses spleen cells derived from tumor challenged or control mice were stimulated in vitro with spleen cells of untreated syngeneic donor mice which had been infected by UV-inactivated (300 mJ) rVV-AFP or, as a negative control for specificity, rVV-pSC11 at a multiplicity of infection (MOI) of five and then irradiated with 20 Gy (2000 rad) to prevent stimulator cells from proliferation. Subsequently, a 6-hour 51 Cr release assay was performed. As target cells AFP expressing syngeneic Hepa1-6 cells and AFP negative syngeneic Lewis lung carcinoma cells were used. Results were expressed according to the formula: % lysis=(experimental release−spontaneous release)/(maximum release−spontaneous release). Experimental release. Experimental release represents the mean counts per minute released by target cells in the presence of effector cells. Total release represents the radioactivity released after total lysis of target cells with 5% Triton X-100. Spontaneous release represents the radioactivity present in medium derived from target cells only. Hepa1-6 tumor or MAFP specific lysis was presented by subtraction of lysis values against 3LL from lysis values against Hepa1-6 targets.

IFN-γ and IL-4 ELISPOT assays. Multiscreen-HA 96-well filter plates were coated with 4 μg/ml rat anti-mouse IFN-γ or rat anti-mouse IL-4 antibody (PharMingen, San Diego, Calif., USA, clone R46A2 or 18191 A, respectively) at 4° C. overnight. Spleen cells (1×10⁵/well) derived from tumor challenged or unchallenged mice were cultured in triplicates for 20 hours with 1×10⁴ irradiated stimulator cells (Hepa1-6, 3LL, or rVV-AFP infected spleen cells) per well in 200 μl medium. After culture, the cells were washed out and 2 μg/ml biotinylated rat-anti-mouse IFN-γ or IL-4 antibody (PharMingen, San Diego, Calif., USA, clone XMG1.2 or 18042D, respectively) was added, and the plates were incubated for 3 hours at room temperature. The plates were again washed, incubated with a 1:1000 dilution of Streptavidin-Alkaline Phosphatase polymer (Mabtech, Köln, Germany) for 30 minutes at room temperature and then developed with ALP conjugate substrate solution (BCIP/NBT, BioRad, Richmond, USA). The spots in each well were counted under a microscope, and the values are expressed as numbers of spot-forming cells relative to the number spleen cells added to each well at the start of the culture. As a control for specificity spleen cells of tumor challenged mice and the different irradiated stimulator cells were cultured alone.

Experimental Design of In Vivo Tumor Experiments

Tumor protection studies. C57L/J mice were inoculated with 1×10⁷ Hepa1-6 (groups 1 and 2) or HepaIRF-1hER (groups 3 and 4) cells into the right flank. Group 5 was not inoculated with any tumor. The different groups were treated as follows:

Group 1: No treatment (n=3)

Group 2: 1.5 mg β-estradiol every 2 days i.p. (n=3)

Group 3: no treatment (n=8)

Group 4: 1.5 mg β-estradiol every 2 days i.p. (n=8)

Group 5: 1.5 mg β-estradiol every 2 days i.p. (n=2)

Protection against rechallenge. Naive C57L/J mice or E2 treated animals which had been protected against HepaIRF-1hER tumor growth were rechallenged with 1×10⁷ wild-type Hepa1-6 (n=3) or HepaIRF-1hER (n=3) tumor cells 11 days after stopping E2 treatment (28 days after the initial tumor inoculation). Importantly, these mice did not receive any further E2 treatment.

Tumor therapy. C57L/J mice were inoculated with 1×10⁷ HepaIRF-1hER (groups 1 and 2) cells into the right flank. Both groups did not receive any E2 treatment. At day 19 tumors had reached a size of about 2000 mm³ in both groups. Starting at day 19 group 2 (n=6) was injected i.p. with 1.5 mg β-estradiol every two days until day 28. Subsequently, E2 treatment was stopped again in two mice whereas the remaining four mice were maintained on E2 therapy. Mice of group 1 (n=3) did not receive E2 treatment and were subsequently sacrificed if tumors had reached 10.000 mm³.

In vivo monoclonal antibody ablation. CD4 and CD8 T cell subpopulations were depleted by i.p. injection of purified hybridoma supernatant. A total of 1 mg per mouse per injection of anti-CD8 (clone YTS 169) or anti-CD4 (clone YTS 191.1) (45, 46) was injected on days 5, 3, and 1 before HepaIRF-1hER tumor inoculation and every 5 days thereafter. The different groups were treated as follows:

1. 1.5 mg β-estradiol every 2 days i.p., no depletion (n=2)

2. 1.5 mg β-estradiol every 2 days i.p., CD8 depletion (n=2)

3. 1.5 mg 4 β-estradiol every 2 days i.p., CD4 depletion (n=2)

4. no treatment, no depletion (n=2)

Statistical Analysis. All data were analyzed by Wilcoxon's signed rank test. A two-sided p value of less than 0.05 was considered statistically significant. Tumor appearance and growth to 2500 mm³ was calculated by the Kaplan-Meier method, presented as standard deviation of the mean for each group, and differences between immunized and control mice were calculated by the Mantel-Haenszel test.

### Results

### In Vitro Analysis

IRF-1 Inhibits Cell Growth of the HCC Cell Line Hepa 1-6

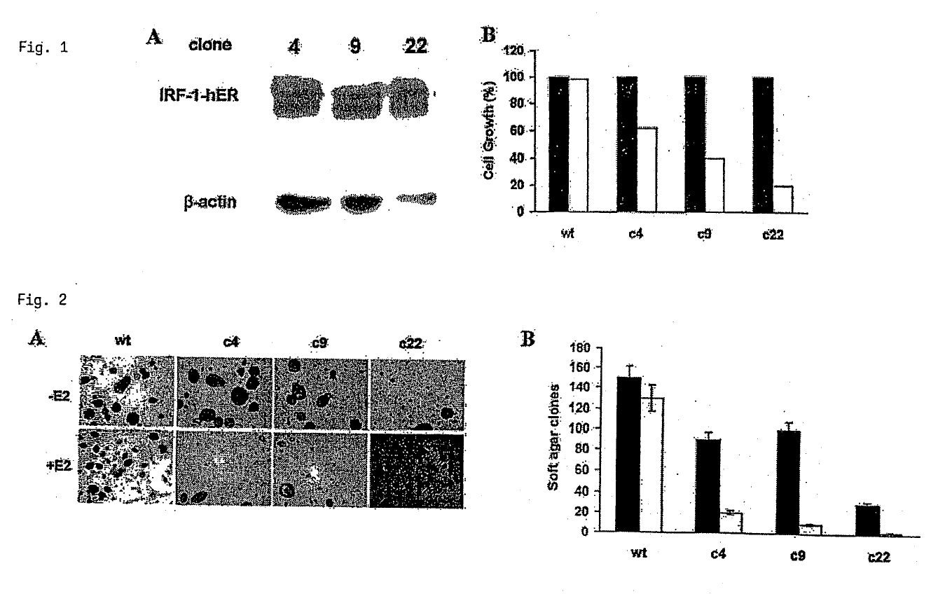

Constitutive expression of IRF-1 imposes a strong cell growth inhibition to several cell lines (4, 24, 40). This results in stable transfectants which are selected for very low, often instable expression of the heterologous IRF-1. To determine the activity of IRF-1 as a growth inhibitor of the HCC cell line Hepa1-6, we, therefore, used a conditionally active IRF-1HER fusion protein. It has been demonstrated that in the absence of hormon stimulation, constitutively expressed chimeric proteins are inactive but can change to an active conformation upon binding of E2 to the hER part of the protein (4, 17, 24). IRF-1-hER was stably transfected into Hepa1-6 cells. Different levels of IRF-1hER expression were observed in the transfectants (FIG. 1A, top panel) and normalized to actin expression (FIG. 1A, bottom panel). Three cell clones with different strength of IRF-1hER expression were selected (c4, c9, c22). Cell growth of these clones was determined 7 days after IRF-1 activation. As shown in FIG. 1B, cells derived from the three cell clones were sensitive to IRF-1 activity with respect to growth inhibition. The extent of proliferation inhibition varied between 40 and 80% between the different cell clones and correlated with the different expression amounts of IRF-1. Wild-type Hepa1-6 cells not expressing IRF-1-hER were used as a control. The nontransfected cell line did not respond to β-estradiol with alterations in cell growth. This indicates that all three cell clones expressed activatible IRF-1 and responded to the typical growth inhibitory properties of the IRF-1 phenotype. It should be, however, noted that growth inhibition of the Hepa1-6 cells was not very strong, if compared to other cell lines (24). Despite the reduction in cell proliferation the cells could be cultivated for a considerable time in this state. Furthermore, these cells did not show any signs of apoptosis upon IRF-1 activation by β-estradiol. This was confirmed by examination of subdiploid DNA ((47) and data not shown) and Annexin staining ((48) and data not shown).

Activated IRF-1 Induces IFN Secretion

The induction of IFN-β is a typical property observed after IRF-1 activation. The amount of secreted IFN-β can be taken as a measure of IRF-1 activity (17). Since IFNs are relevant for immunomodulation, the secretion of IFN-β was determined by an antiviral assay (table 1). IRF-1 was shown to be activated by β-estradiol in all three cell clones. The highest IFN secretion was shown by clone 22, which is in agreement with the strength of IRF-1 expression (FIG. 1A) and proliferation inhibition (FIG. 1B). Using neutralizing antibodies directed against IFN-β the inventors confirmed that the secreted antiviral activity was exclusively IFN-β. IFNs are known to inhibit cell proliferation. However, the amount of IFN-β secreted by the Hepa1-6 cell clones is not sufficient to mediate the observed effect on cell growth as determined by the treatment of control cells with comparable amounts of recombinant murine IFN-β (data not shown).

Decreased Anchorage-Independent Growth During IRF-1 Activation

The most definitive in vitro characteristics distinguishing tumorigenic cells from nontumorigenic cells is anchorage-independent growth. To determine whether IRF-1 reverses the transformed phenotype of this HCC cell line in vitro, the inventors tested its ability to form anchorage-independent colonies in the presence of inactive or activated IRF-1 (FIG. 2). The untransfected tumor cell clones grew well in soft agar. The colony formation of the transformed wild-type Hepa1-6 cell line not expressing IRF-1hER was not influenced by β-estradiol. In contrast, the ability of soft agar growth was significantly decreased by the activated IRF-1 in the different cell clones. In contrast to untransfected cells the IRF-1hER bearing cells formed fewer but somewhat bigger colonies. In presence of E2, clone 22 formed the lowest amount of colonies in soft agar which inversely correlates to the strength of IRF-1hER expression. Clone 9 showed the highest ratio of soft agar colony formation from untreated over E2 treated cells. Therefore, clone c9, in following simply designated HepaIRF-1hER, was used for further in vitro characterization and for the in vivo tumor model.

Activated IRF-1hER Modulates Immunologically Relevant Cell Surface Protein Expression

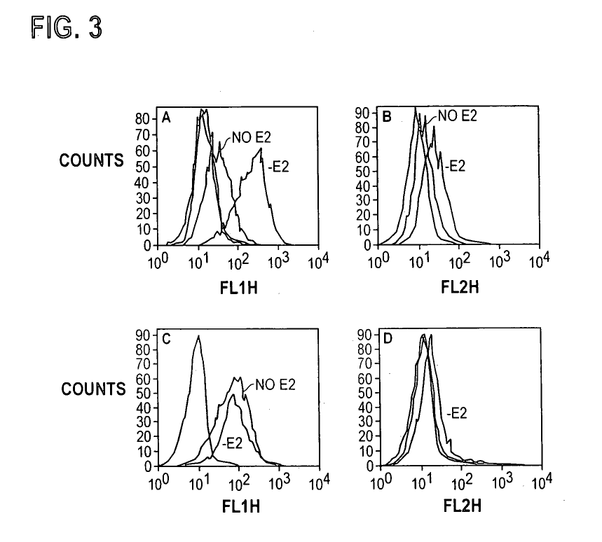

IRF-1 expression has been previously shown to increase expression of MHC genes (7, 31). The inventors examined the levels of MHC class I, MHC class II, CD54, CD80, and CD86 expression on the cell surface before and after IRF-1 activation with β-estradiol by FACS analysis. Wild-type Hepa1-6 and E2 untreated HepaIRF-1hER cells were characterized by the lack of MHC class II, CD80, and CD86 expression. MHC class I (H-2K^{b}/H-2D^{b}) was expressed at low and CD54 at high levels. E2 treatment of HepaIRF-1hER cells resulted in a strong upregulation of H-2K^{b}/H-2D^{b}, CD54 expression remained unchanged, and MHC class II expression was weakly upregulated (FIG. 3). CD80 and CD86 expression remained negative (data not shown).

Adenoviral Mediated Expression of IRF-1 Leads to Strong IFN-β Secretion of HCC Cells

To tranfer IRF-1 in a wide variety of the cells and as a delivery system for in vivo transduction adenoviral vectors based on pAdcos45 containing expression cassettes for the IRF-1-hER fusion protein were constructed. The cDNA of IRF-1-hER was introduced into the adenoviral vector pAdcos45 and viruses were prepared.

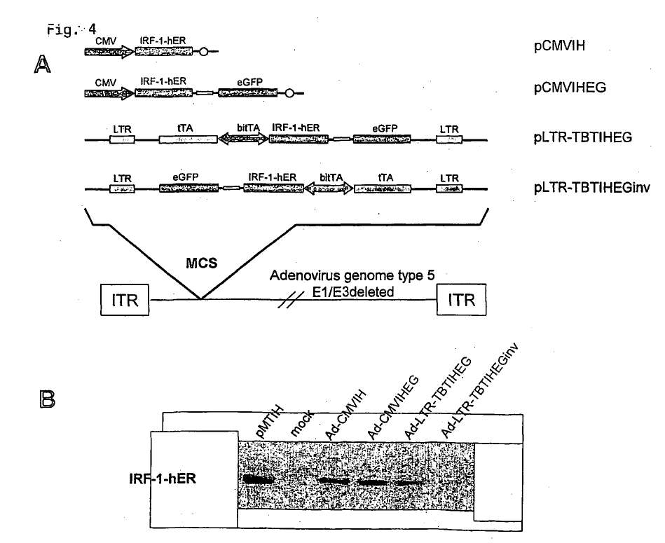

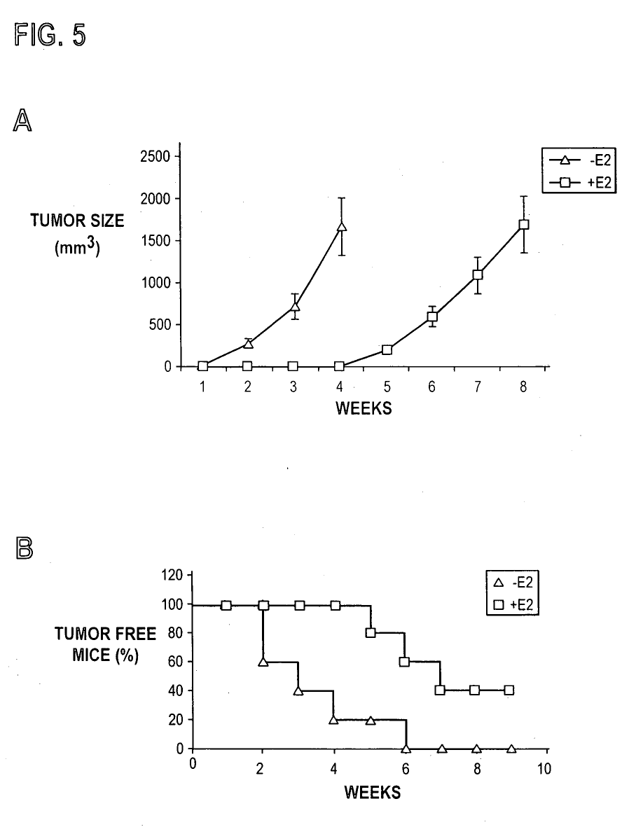

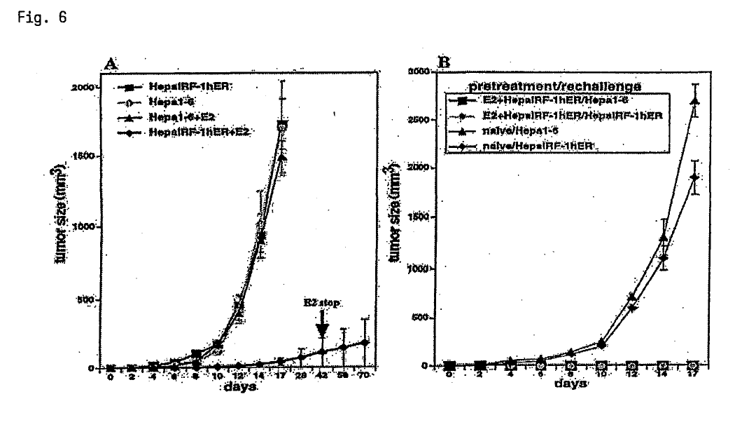

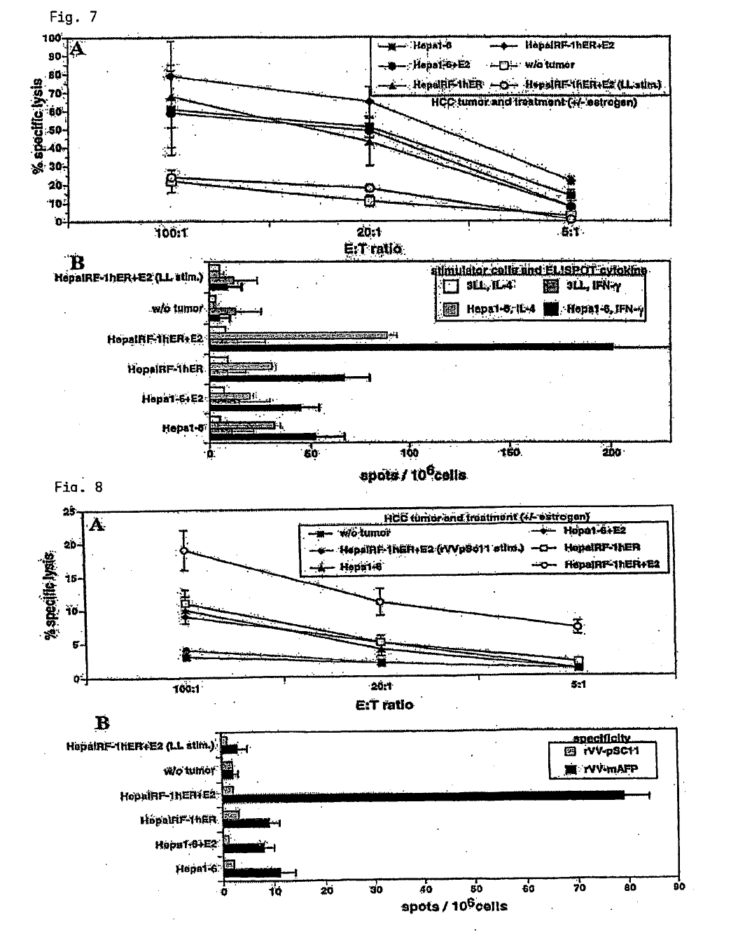

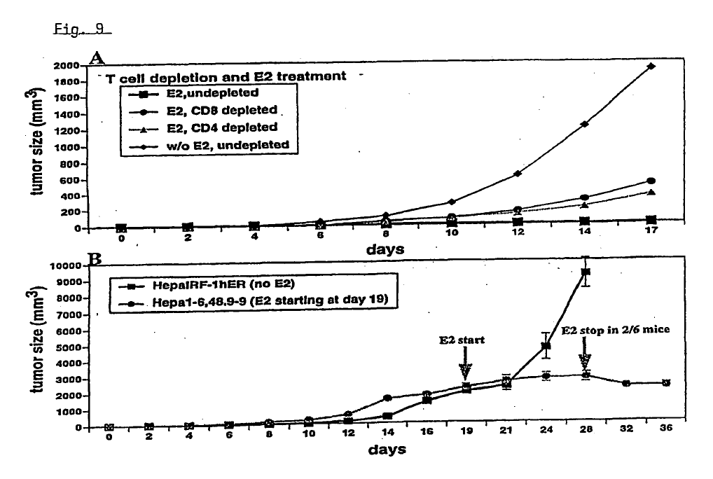

Hepa1-6 cells were infected with either pAd-IH, pAd-IHEG, pAd-LTR-TBTIHEG and pAd-LTR-TBTIHEGinv viruses (FIG. 4A). IRF-1-hER expression was analysed by Western blot analysis 48 h after infection. High levels of IRF-1hER expression was found in cells infected with these adenoviruses (FIG. 4B). IFN secretion was measured after E2 treatment or mock treatment of the infected cells. IFN was detected in the culture supernatants of cells infected with adenovirauses containing the IRF-1 hER gene after E2 treatment, indicating that only IRF-1 activation but not the infection as such induces IFN secretion (Table 2). The amount of secreted IFN in adenovirally transduced cells was 3,5-6 fold higher than from transfectants which stably express the IRF-1hER fusion protein.

IRF-1 Effects in Oncogenically Transformed NIH3T3 Cells

The effects of IRF-1 in oncogenically transformed NIH3T3 cells were examined. NIH3T3 cells conditionally expressing the oncogenes c-myc and c-Ha-ras were used. Both oncogenes were bicistrinically expressed under the control of the tetracycline regulatable promoter (Kröger, Dissertation, Universität Braunschweig, 1999). The cells were were stably transfected with the IRF-1-hER fusion protein encoding construct. This cell line allows the investigation of IRF-1 effects in the non-transformed NIH3T3 cells (presence of Doxycycline) and in the transformed state of the cells (absense of Doxycycline). The antitumoral activity of IRF-1 in vitro was investigated: Influence on proliferation, soft agar growth and IFN induction with and without IRF-1hER activation by E2 treatment were measured. This was done in the non-transformed as well as in the transformed state. E2 and Doxycyline were added for five days. Activation IRF-1-hER led to marked reduction of cell growth in both, non transformed and transformed cells to the same level, despite the fact that transformed cells in the absence of E2 showed enhanced cell growth (Table 3).

To determine wether IRF-1 reverses the transformed phenotype of the cells to the formation of colonies in soft agar was assayed. The cells in the non-transformed status did not grow in soft agar. In contrast, in the absence of Doxycycline (transformed status) the cells grew well and formed soft agar clones. The ability of soft agar growth was completely abolished by the activation of IRF-1 by E2 gift (Table 3).

IFN secretion as determined by an antiviral assay was taken as a measure of IRF-1 activity. IRF-1 activation by E2 treatment was demonstrated to be equivalent in non-transformed and transformed cells (Table 3).

IRF-1 Activation Decreases Tumor Growth of Transformed Cells in Nude Mice

As IRF-1 inhibited cell transformation in vitro in oncogenically transformed NIH3T3 cells its effects on tumorigenicity in vivo were assessed. To verify the possible anti-tumor activity of IRF-1, the cells were injected subcutaneously into the flanks of nude mice. Tumor formation was assayed. Injection of the transformed cells led to tumor formation within four weeks and 100% of implanted animals developed a tumor within six weeks (FIG. 5). If mice were inoculated with transformed cells and were treated with E2, the kinetics of tumor growth was dramatically changed. Tumor sizes up to 1500 mm³ were reached four weeks later than in untreated animals and 40% of the E2 treated mice developed no tumors. These results demonstrate that activation of IRF-1hER fusion protein is sufficient to prolong kinetics of tumorigenicity in transformed cells in animals lacing T- and B-cells.

### In Vivo Analysis

IRF-1 Activation Inhibits HCC Growth

To determine the antitumoral efficacy of IRF-1hER expression against murine Hepa1-6 HCCs growing subcutaneously in C57L/J mice, different treatment groups were randomly designed. The Hepa1-6 HCC cell line used for the tumor model in syngeneic C57L/J mice was characterized by moderately fast tumor growth and 100% of implanted animals developed a tumor. Both wild-type Hepa1-6 and HepaIRF-1hER cells exhibited nearly identical tumerogenicity after s.c. injection in vivo suggesting the presence of an inactive IRF-1hER fusion protein in E2 untreated HepaIRF-1hER cells (FIG. 6A, p>0.5). Within 17 days large tumors developed with an average size of 1500 mm³. If mice inoculated with wild-type Hepa1-6 cells were treated with E2 no effect on tumor development was observed in comparison to mice challenged with Hepa1-6 or HepaIRF-1hER cells without E2 treatment. These results demonstrate that the E2 treatment itself has no negative effect on tumor growth and animal health. If C57L/J mice inoculated with HepaIRF-1hER, however, received 2-daily i.p. injections with E2 starting at the time of tumor inoculation, tumor growth was significantly suppressed. It was an important finding that six out of eight animals were completely protected against tumor growth and two animals developed only very small tumors which were characterized by a slow growth rate (FIG. 6A). After 40 days the tumor size was only 450 mm³ and stopping E2 treatment at day 42 did not result in a faster growth rate of the tumor.

IRF-1 Activation in Tumor Cells Induces T-Cell Memory

The inventors were interested to investigate the presence of tumor specific memory T cells in E2 treated mice protected against challenge with HepaIRF-1hER. Therefore, tumor free mice were inoculated with 1×10⁷ wild-type Hepa1-6 (n=3) or HepaIRF-1hER cells (n=3) 28 days after tumor challenge and 11 days after stopping E2 treatment. Importantly, these mice did not receive any further E2 treatment. As demonstrated in FIG. 6B all mice in both groups were protected against tumor growth but not after CD8+ T cell in vivo depletion (n=2, data not shown). In contrast, naive C57L/J mice without E2 treatment were characterized by rapid Hepa1-6 and HepaIRF-1hER tumor growth. These results suggest the presence of tumor specific T cell memory after primary priming of tumor specific immunity by the expression of active IRF-1hER.

Induction of CTL Activity through IRF-1 Activated Tumor Cells

In fact, strong CTL activity against Hepa1-6 target cells after in vitro stimulation using irradiated Hepa1-6 cells was observed in mice challenged with HepaIRF-1hER cells and treated with E2 (FIG. 7A). This CTL activity was specific against Hepa1-6 tumor cells because spleen cells derived from mice which were not tumor challenged or from E2 treated mice challenged with HepaIRF-1hER cells and in vitro stimulated with 3LL cells displayed only weak background killing activity against Hepa1-6 targets (FIG. 7A). Primary T cell responses were evaluated by monitoring cytokine-producing cells in vivo. A significant increase in the number of spleen cells secreting IFN-γ (1 in 5,000) and IL-4 (1 in 10,000) (FIG. 7B) upon stimulation with irradiated Hepa1-6 cells was observed in E2 treated mice inoculated with HepaIRF-1hER tumors in comparison to E2 untreated/HepaIRF-1hER, E2 untreated/Hepa1-6, or E2 treated/Hepa1-6 challenged mice (1 in 20,000 IFN-γ and 1 in 40,000 IL-4 secreting T cells, p=0.01) suggesting significant development of both TH1 and TH2 tumor immunity. In contrast to the in vivo results obtained by ELISPOT analysis, the differences of CTL in vitro killing activity in the ⁵¹Cr-release assay (FIG. 7A) were not statistically significant between the different groups. This may be the result of in vitro expansion of tumor specific T cells and the lower sensitivity of the ⁵¹Cr-release assay in comparison to the ELISPOT technique.

IRF-1 Activation Breaks Ignorance to Tumor Specific Self Antigens

To determine immune responses against HCCs in more detail and to find out if expression of activated IRF-1 was able to prime immune responses against a tumor specific antigen the inventors chose the HCC specific self antigen AFP which is frequently expressed at high levels in HCC cells as a target. Intermediate CTL activity against Hepa1-6 HCCs endogenously expressing AFP at high levels was observed in E2 treated/HepaIRF-1hER challenged mice (FIG. 8A). CTL activity was significantly stronger (p=0.02) and number of AFP specific IFN-γ (FIG. 8B) producing spleen cells (1 in 11,000) was higher (p=0.001) in these mice in comparison to the other groups (1 in 1,000,000). In control animals without tumors no specific CTL activity or enhanced background lysis against Hepa1-6 or 3LL target cells was observed. In addition, no increased lysis of these target cells was seen after in vitro stimulation of effector cells with rVV-pSC11 derived from E2 treated mice challenged with HepaIRF-1hER cells suggesting specificity of CTL activity against AFP (FIG. 8A). Performing ELISPOTs using rVV-AFP infected spleen cells alone without effectors or using effectors alone did not result in increased background spot formation, additionally suggesting AFP specificity (data not shown). These data demonstrate that tolerance to the self antigen AFP can be broken by intratumoral expression of activated IRF-1, a mechanism presumably participating in HCC tumor growth control.

Other Factors than Host Immune Response Participate in Rejection of IRF-1 Activated Tumor Cells

To determine if the host antitumoral immune response is the only parameter responsible for tumor rejection, in vivo T cell depletion experiments were performed in E2 treated mice challenged with HepaIRF-1hER tumors (FIG. 9A). Undepleted mice were again protected against tumor growth. Both CD4 and CD8 T cell depletion resulted in tumor growth in all mice which, however, was significantly delayed in comparison to undepleted mice which did not receive E2 treatment. This finding implies that the host immune response is an important factor in tumor protection but seems to be only partial in the initial control of tumor growth.

### IRF-1 Activation Stops Increase of Actively Growing Tumors

To assess therapeutic efficacy of IRF-1hER activation against HepaIRF-1hER HCCs growing subcutaneously in E2 untreated C57L/J mice, E2 treatment was started at day 19 after tumor challenge. At this point in time tumors had reached an average size of about 2000 mm³. As demonstrated in FIG. 9B, tumor growth was permanently arrested as early as four days after starting of E2 treatment demonstrating a significant therapeutic potential of IRF-1 activation against HCCs. By contrast, HepaIRF-1hER tumors grew rapidly in E2 untreated mice. E2 treatment for nine days was sufficient to induce long term tumor growth control. This was readily demonstrated in two out of six E2 treated mice which E2 treatment was stopped at day 28 again. Tumor growth in these mice did not differ as compared to mice which continued to receive two daily E2 injections.

IRF-1 Activation in Tumors on the one Flank Influences Tumor Growth in Both Flanks

The efficacy of IRF-1 activation as a therapeutic vaccine against tumors was examined. Mice were injected subcutaneosly with HepaIRF-1hER cells in the right flank and wt Hepa1-6 cells into the left flank. The animals were either treated with 1.5 mg E2 every two days or were left untreated. Activation of IRF-1-hER abolished tumor growth of HepaIRF-1hER cells on the right flank of the mice. In addition, although tumor growth of wt Hepa1-6 cells on the left flank was also decreased in comparison to Hepa1-6 tumor growth which was initiated in the first days, a further expansion as it took place in untreated animals was inhibited.

### Discussion

HCC is a highly malignant tumor with a poor prognosis and few therapeutic options. A new immunotherapeutic approach aimed at the activation of IRF-1 was examined. A murine HCC cell line (HepaIRF-1hER) encoding an IRF-1/human estrogen receptor fusion protein, which becomes active in the presence of β-estradiol, was constructed. The in vitro phenotype, cell growth, anchorage-independent growth in vitro, immunogenicity in vivo, and the therapeutic potential of IRF-1 all were examined. Stable constitutive IRF-1 expression has the disadvantage to select for low expressing clones. The actual transgene expression in such cell lines does not much override endogenous IRF-1 expression. Furthermore, constitutive expression induces a selection towards loss of IRF-1 responsiveness over time (4, 40). In contrast, the activatible IRF-1hER system used in the invention allows the tight regulation of IRF-1 activity and to express rather high levels of IRF-1 in the tumor cells. The E2 (E2=β-estradiol) activatible system has been extensively studied and also compared with the tetracycline regulated transcription activation of the wild type IRF-1 gene. No differences were found (Kirchhoff et al., 1995; Köster et al., 1995). Because of the use of a mutant estradiol receptor gene (49) the fusion protein is insensitive to low estradiol concentrations and thus can be used in mice without being activated by endogenous estrogen levels. A respective tamoxifen specific mutant of IRF-1hER* (50), however, did not show tight regulation of IRF-1 activity (A. Kröger, unpublished). Using the β-estradiol inducible system we, therefore, could address the antitumoral effects of IRF-1 in more detail and dissect the activation of its different antitumoral effector arms.

Inhibition of growth and transformation of Hepa1-6 cells was demonstrated in vitro confirming a property which has to be attributed to the innate immune activity of IRF-1. Ectopic IRF-1 expression suppresses cellular transformation properties in vitro induced by different oncogenes, such as myc, fosB (29), IRF-2 (51, 52), EGFR and E1a/b (24). In accordance with results obtained earlier with embryonic fibroblasts from IRF-1^{-/-} mice in which expression of c-Ha-ras oncogene in wild type cells but not in IRF-1^{-/-} cells forces the cells to undergo apoptosis under growth restricted conditions (Tanaka et al., 1994), the combined activity of EGFR and IRF-1 in NIH3T3 cells was shown to induce significant cell death by apoptosis (24). However, the data presented in this report indicate that oncogene dependent tumor suppression in vitro is not necessary to be mediated by apoptosis. Thus, other mechanisms of transformation inhibition are expected to act. IRF-1 exerts its effects by DNA-binding and transactivation of a number of genes which might contribute to transformation inhibition. Among them are Lysyl oxidase (16), PKR (17, 53), 2′-5′ OASE (18), Indoleamine 2,3-dioxigenase (19), and Angiotensin type II receptor (20). The role of IFN-β secretion in this context is not clear but might act as a feed-back enhancer of these genes.

IRF-1 has several other relevant in vivo antitumor activities. These are due to the immunomodulatory effects of IRF-1, such as the stimulation of helper T and NK cells (54, 55), transcriptional enhancement of MHC genes (4, 31, 56, 57) and of genes involved in antigen presentation (38, 58). Thus, most events or drugs which enhance the expression or activity of IRF-1 might be useful in cancer therapy by inducing specific killing of transformed cells. Indeed, experiments in mice have been described demonstrating that expression of IRF-1 in aggressive nonimmunogenic sarcoma cells suppresses the malignant phenotype (40).

The suppression and control of a highly tumorigenic HCC cell line in vivo were the most important activities of IRF 1. E2 treatment protected 75% of mice against challenge with the HepaIRF-1hER tumor and mice developing tumors were characterized by a significant suppression of tumor growth and enhanced survival as compared to E2 untreated animals. Similar results were observed in a recent study where the constitutive expression of IRF-1 in a sarcoma cell line resulted in partial tumor control (40). By T cell depletion experiments it was demonstrated that CD4+ and CD8+ T cells play an important role in control of tumor growth confirming the known importance of IRF-1 in induction of TH1 differentiation (55). In addition, the inventors observed a significant activation of tumor specific TH2 cells which may synergistically act against tumor growth (59, 60). The effect of both CD4+ and CD8+ depletion experiments cannot explain the whole effect of IRF 1 mediated tumor growth control. It was shown that IFN-β can activate NK cells. Therefore, it is possible that NK cells contribute to tumor control.

More importantly, it was demonstrated for the first time that intratumoral expression of IRF-1 induces significant T cell responses against a tumor associated antigen such as the HCC specific self antigen AFP suggesting that tolerance towards AFP can be broken by this approach. AFP specific CTL activity was low as compared to highly immunogenic viral antigens, such as HBV or HCV structural proteins (61, 62). This may be the result of the low CTL precursor frequencies and/or low affinity TCRs. Recent studies, however, demonstrated that AFP specific T cells after DNA- or dendritic cell-based immunization are functional in vivo against AFP expressing murine HCCs (44, 63) suggesting that AFP specific T cells contributed to the antitumoral effects as presented in this invention. Although cellular immunity alone was not able to completely control tumor growth as demonstrated by the in vivo depletion experiments, significant T cell memory against the tumor was induced which protected mice against a rechallenge with HepaIRF-1hER and even wild-type Hepa1-6 cells without further E2 treatment. More important, the potential therapeutic efficacy of IRF-1 expression reflecting the clinical situation after HCC diagnosis was shown. Treatment of mice bearing large HepaIRF-1hER tumors with E2 resulted in a growth arrest of the tumor within four days. E2 treatment over a period of nine days was sufficient to control tumor growth long term without any further E2 treatment. According to the tumor protection studies described above this long term tumor control may be primarily immune mediated.

Enhanced immunogenicity of HCC tumors expressing IRF-1 may be mediated by upregulation of MHC class I and II molecules as previously described, though MHC class II induction was low in HCC cells. The costimulatory molecules CD80 and CD86 could not been detected suggesting that priming of antitumoral immune responses must have occured in the draining lymph nodes by professional antigen presenting cells. Additional factors involved in IRF-1 mediated tumor growth control may be the increased generation of MHC class I restricted antigenic peptides for presentation to the immune system by proteasomes (38, 39, 58).

As predicted by the Yim et al. (40) it was shown for the first time that ectopic IRF-1 expression induces significant therapeutic antitumoral immune responses and primes immunity against a tissue specific self tumor antigen, e.g. AFP. Therefore, the inventors results may have implications for local and AFP-based immunotherapy of HCC.

Summing up, hepatocellular carcinoma (HCC) is a highly malignant tumor with a poor prognosis and few therapeutic options.

HCC is regarded as an example for other tumor entities. There is good evidence that IRF-1 will have the same systemic effects in other tumor species. The following examples support this hypothesis:

1. Yim et al. (1997; 40) reported that a sarcoma cell line when expressing IRF-1 partially induced rejection and immunity. The effects are not complete because IRF-1 was not sufficiently expressed.

2. The example with oncogenically transformed 3T3 cells supports the version that tumor growth is significantly reduced when active IFR-1 is expressed, even in nude mice. The delay of the tumor growth in nude mice is much higher than it was expected from in vitro data, indicating that the remaining part of the immune system in these animals has been involved.

3. In vitro data with a number of different oncogenically transformed cell lines show that IRF-1 can significantly or completely suppress soft agar growth, induce interferon secretion, and activate MHC upregulation. (Kirchhoff et al., 1999, and Kröger et al., unpublished data).

Although detailed molecular function of tumor defence in mice is not known, the presented data indicate that immune cells (CTL, TH1 and TH2 cells) are involved. The nude mice experiments suggest that NK cells are also involved. It is known that IFN-β is a strong activator of NK-cell activity. Further IFN-β is also known for diverse activation functions in the adaptive immune system. Finally, apoptosis as well as growth inhibitory effects of IRF-1 might contribute to the tumor inhibitory effect which is seen in the cells overexpressing IRF-1. While immunological effects are thought to be mediated by MHC upregulation and interferon secretion other, as yet unknown activities induced by IRF-1 could contribute to the antitumor action.

The interferon which is secreted upon IRF-1 activation could act systemically as well as local. It is thus possible that the production of IFN in the surrounding of the antigen presenting cells (those ones overexpressing IRF-1) lead to high stimulation of the antitumor activities.

In the invention work IRF-1 was demonstrated to induce antitumor activities. It is well known that other IRFs have similar binding properties if compared to IRF-1. E.g., IRF-3 seems to be able to activate the same or a similar set of genes that are activated by IRF-1. Thus, permanently activated IRF-3 or IRF-3 variants, which are constitutively active could have the same antitumor activity as demonstrated here.

For convenience of the observed effects in the animal tumor model to human therapy, all scenarios are based on the strong activity of IRF-1 or related transcription factors (see above).

1. Cellular Therapies

Tumor cells of the patients or tumor cells from other patients with the same tumor entity (allogenic cells) could be loaded with genes by virus infection or other gene transfer methods. It is unimportant if these genes are expressed only transiently or for a long time period. Alternatively, non-specific cells or professional antigen presenting cells which are able or forced to present tumor antigens which are relevant for the target tumor would be loaded by IRF constructs. These cells could be derived from the patient or could be allogenic. The activation of the IRF genes in those cells could be either activated in vitro immediately before the gift to the patient. Alternatively, they might be activated within the patient by gift of respective activators. (β-estradiole or tetracyclines). In humans, cellular therapies are usually carried out by inactivating other cells, by methods like UV or gamma irradiation.

2. Gene Therapies by Viral Transfer Methods

IRFs, like those ones described above could be transduced by viral vectors like the described adenoviral vector. Preferentially, this would be done by infection of the viruses into the tumor or into tissue close to the tumor. In certain cases it could also be done by systemic application. This would be typically done in the case of liver tumors by application of adenoviruses into the blood stream. It is well known that adenoviruses are mainly captured in liver and gene transfer would be thus far liver-specific. The activation or reduction of the IRF harboured by the viral vectors would be activated in vivo in the patients by respective agents. It should be mentioned that earlier work has shown that IRF-1 activation in non-tumor cells causes reversible proliferation inhibition but does not lead to determental effects.

3. Gene Therapy by Non-Viral Methods

A number of methods by which genes could be transferred into human tissue are known. Amongst them is lipofection, gene gun, electroporation and others. IRF's could be transfected by these methods into the respective tumor tissues.

4. Activation or Induction of Endogenous

IRF-1 in the patients' tumor or antigen presenting cells could be activated or induced. From a number of compounds like cytokines and other biological response modifiers it is known that they activate the transcription and production of IRF-1. Strong inducers or combinations of such could be used to induce IRF-1 and to induce the observed antitumor effects. Other compounds typically found by high throughput screening which would activate endogenous IRF-1 could be used in the same way.

TABLE 1
IRF-1 activation leads to IFN secretion^{c}

| | IFN secretion | | |
|---|---|---|---|
| | (IU/ml) | | |
| | | | +E2^{a} + anti-IFN- |
| Clone | −E2 | +E2^{a} | Ab^{b} |
| wt | n.d | n.d | n.d |
| c4 | n.d | 125 | n.d |
| c9 | n.d | 125 | n.d |
| c22 | n.d | 180 | n.d |

n.d: not detectable
^{a}1 μM β-estradiol
^{b}anti-IFN-β antibody neutralizing 500 IU
^{c}Hepa1-6 cells and HepaIRF-1hER cell clones were treated with 1 μM of β-estradiol for five days. Accumulated IFN was measured in the supernatant as described in Material and Methods and normalized to 10⁶ cells.

TABLE 2a
Titer of adenoviral productions

Viral Titer (viral particles/ml)

| Virus | −E2 | +E2^{a} |
|---|---|---|
| AdCMVIRF-1 | 0-1 × 10⁴ | 0-1 × 10⁴ |
| AdCMVIH | 5 × 10⁹-1.5 × 10¹⁰ | 0-1 × 10⁴ |
| AdCMVIHEG | 5 × 10⁹-1 × 10¹⁰ | —^{b} |
| Ad-LTR-TBTIHEG | 4 × 10⁸-1 × 10⁹ | — |
| Ad-LTR-TBTIHEGinv | 3 × 10⁸-1 × 10⁹ | — |

^{a}during production, 293 cells were treated with 1 μM β-estradiol
^{b}not measured

TABLE 2b
IFN secretion in cells infected with IRF-1-hER containing
adenoviruses after β-estradiol treatment

| t | |
|---|---|
| transduced adenoviral | IFN secretion (IU/ml) |

| construct | −E2 | +E2^{b} |
|---|---|---|
| mock | nd^{c} | Nd |
| Ad-CMVIH | nd | 5000 |
| Ad-CMVIHEG | nd | 5000 |
| Ad-LTR-TBTIHEG | nd | 5000 |
| Ad-LTR-TBTIHEGinv | nd | 2500 |

^{a}Hepa1-6 cells were 24 h after infected with adenovirus treated with 1 μM E2 for 24 h. Accumulation of IFN was measured in the supernatant and normalized to 10⁶ cells.
^{b}1 μM β-estradiol
^{c}not detectable

TABLE 3
Phenotype of IRF-1 activation in NIH3T3TA/IH/MR cells

| | |
|---|---|
| +DOX^{a} | −Dox |

| | −E2 | +E2^{b} | −E2 | +E2^{b} |
|---|---|---|---|---|
| Proliferation^{c} | 100 | 60 | 130 | 65 |
| (%) | | | | |
| Soft agar | 0 | 0 | 130 | 0 |
| growth^{d} | | | | |
| (number of | | | | |
| clones) | | | | |
| IFN secretion^{e} | nd^{f} | 750 | nd | 750 |
| (IU/ml) | | | | |

^{a}2 μg/ml Doxycyclin
^{b}1 μM β-estradiol
^{c}The metabolic activity as a measure for cell growth of the cells were measured seven days after treatment
^{d}Anchorage independent growth of the cells in soft agar. The number of colonies were determined two weeks after treatment.
^{e}Accumulation of IFN was measured five days after treatment in the supernatant and normalized to 10⁶ cells.
^{f}not detectable

TABLE 4
IRF-1 activation in tumors on the one flank influences tumor growth
in both flanks

Tumor volume (mm³)
(mean ± SD)

| cell type injected^{a} | −E2 | +E2^{b} |
|---|---|---|
| Hepa1-6 (wt) | 1630 ± 596 | 1630 ± 596 |
| HepaIH | 1317 ± 380 | 22,4 ± 179 |
| Hepa1-6 (left) + | 1630 ± 596 | 179 ± 288 |
| HepaIH (right)^{c} | 1630 ± 596 | nd^{d} |

^{a}5 × 10⁶ Hepa1-6 or HepaIRF-1hER cells were inoculated subcutanously in the flank of the mice
^{b}mice were treated with 1.5 mg E2 every two days i.p.
^{c}mice were inoculated with 5 × 10⁶ HepaIRF-1hER cells on the right flank and with 5 × 10^{6 cells on the left flank}
^{d}not detectable

### BIBLIOGRAPHY

1. Bosch, X., Ribes, J., and Borras, J. Epidemiology of primary liver cancer, Seminar Liver Dis. 19: 271-285, 1999.

2. Okuda, K. Hepatocellular carcinoma, J. Hepatol. 32: 225-237, 2000.

3. Harroch, S., Revel, M., and Chebath, J. Induction by interleukin-6 of interferon regulatory factor 1 (IRF-1) gene expression through the palindromic interferon response element pIRE and cell type-dependent control of IRF-1 binding to DNA, Embo J. 13: 1942-9, 1994.

4. Kirchhoff, S., Schaper, F., and Hauser, H. Interferon regulatory factor 1 (IRF-1) mediates cell growth inhibition by transactivation of downstream target genes, Nucleic Acids Res. 21: 2881, 1993.

5. Pine, R. Constitutive expression of an ISGF2/IRF1 transgene leads to interferon-independent activation of interferon-inducible genes and resistance to virus infection, J Virol. 66: 4470-8, 1992.

6. Reis, L. F., Harada, H., Wolchok, J. D., Taniguchi, T., and Vilcek, J. Critical role of a common transcription factor, IRF-1, in the regulation of IFN-beta and IFN-inducible genes, Embo J. 11: 185-93, 1992.

7. Chang, C. H., Hammer, J., Loh, J. E., Fodor, W. L., and Flavell, R. A. The activation of major histocompatibility complex class I genes by interferon regulatory factor-1 (IRF-1), Immunogenetics. 35: 378-84, 1992.

8. Kimura, T., Nakayama, K., Penninger, J., Kitagawa, M., Harada, H., Matsuyama, T., Tanaka, N., Kamijo, R., Vilcek, J., Mak, T. W., and et al. Involvement of the IRF-1 transcription factor in antiviral responses to interferons, Science. 264: 1921-4, 1994.

9. Fujita, T., Kimura, Y., Miyamoto, M., Barsoumian, E. L., and Taniguchi, T. Induction of endogenous IFN-alpha and IFN-beta genes by a regulatory transcription factor, IRF-1, Nature. 337: 270-2, 1989.

10. Harada, H., Fujita, T., Miyamoto, M., Kimura, Y., Maruyama, M., Furia, A., Miyata, T., and Taniguchi, T. Structurally similar but functionally distinct factors, IRF-1 and IRF-2, bind to the same regulatory elements of IFN and IFN-inducible genes, Cell. 58: 729-39, 1989.

11. Matsuyama, T., Kimura, T., Kitagawa, M., Pfeffer, K., Kawakami, T., Watanabe, N., Kundig, T. M., Amakawa, R., Kishihara, K., Wakeham, A., and et al. Targeted disruption of IRF-1 or IRF-2 results in abnormal type I IFN gene induction and aberrant lymphocyte development, Cell. 75: 83-97, 1993.

12. Ruffner, H., Reis, L. F., Naf, D., and Weissmann, C. Induction of type I interferon genes and interferon-inducible genes in embryonal stem cells devoid of interferon regulatory factor 1, Proc Natl Acad Sci USA. 90: 11503-7, 1993.

13. Reis, L. F., Ruffner, H., Stark, G., Aguet, M., and Weissmann, C. Mice devoid of interferon regulatory factor 1 (IRF-1) show normal expression of type I interferon genes, Embo J. 13: 4798-806, 1994.

14. Pellegrini, S. and Schindler, C. Early events in signalling by interferons, Trends Biochem Sci. 18: 338-42, 1993.

15. Kamijo, R., Harada, H., Matsuyama, T., Bosland, M., Gerecitano, J., Shapiro, D., Le, J., Koh, S. I., Kimura, T., Green, S. J., and et al. Requirement for transcription factor IRF-1 in NO synthase induction in macrophages, Science. 263: 1612-5, 1994.

16. Tan, R. S., Taniguchi, T., and Harada, H. Identification of the lysyl oxidase gene as target of the antioncogenic transcription factor, IRF-1, and its possible role in tumor suppression, Cancer Res. 56: 2417-21, 1996.

17. Kirchhoff, S., Koromilas, A. E., Schaper, F., Grashoff, M., Sonenberg, N., and Hauser, H. IRF-1 induced cell growth inhibition and interferon induction requires the activity of the protein kinase PKR, Oncogene. 11: 439-45, 1995.

18. Benech, P., Vigneron, M., Peretz, D., Revel, M., and Chebath, J. Interferon-responsive regulatory elements in the promoter of the human 2′,5′-oligo(A) synthetase gene, Mol Cell Biol. 7: 4498-504, 1987.

19. Takikawa, O., Kuroiwa, T., Yamazaki, F., and Kido, R. Mechanism of interferon-gamma action. Characterization of indoleamine 2,3-dioxygenase in cultured human cells induced by interferon-gamma and evaluation of the enzyme-mediated tryptophan degradation in its anticellular activity, J Biol Chem. 263: 2041-8, 1988.

20. Horiuchi, M., Yamada, T., Hayashida, W., and Dzau, V. J. Interferon regulatory factor-1 up-regulates angiotensin II type 2 receptor and induces apoptosis, J Biol Chem. 272: 11952-8, 1997.

21. Tanaka, N., Ishihara, M., Kitagawa, M., Harada, H., Kimura, T., Matsuyama, T., Lamphier, M. S., Aizawa, S., Mak, T. W., and Taniguchi, T. Cellular commitment to oncogene-induced transformation or apoptosis is dependent on the transcription factor IRF-1, Cell. 77: 829-39, 1994.

22. Lowe, S. W. and Ruley, H. E. Stabilization of the p53 tumor suppressor is induced by adenovirus 5 E1A and accompanies apoptosis, Genes Dev. 7: 535-45, 1993.

23. Attardi, L. D., Lowe, S. W., Brugarolas, J., and Jacks, T. Transcriptional activation by p53, but not induction of the p21 gene, is essential for oncogene-mediated apoptosis, Embo J. 15: 3693-701, 1996.

24. Kirchhoff, S. and Hauser, H. Cooperative activity between HER oncogenes and the tumor suppressor IRF-1 results in apoptosis, Oncogene. 18: 3725-36, 1999.

25. Chin, Y. E., Kitagawa, M., Kuida, K., Flavell, R. A., and Fu, X. Y. Activation of the STAT signaling pathway can cause expression of caspase 1 and apoptosis, Mol Cell Biol. 17: 5328-37, 1997.

26. Tamura, T., Ishihara, M., Lamphier, M. S., Tanaka, N., Oishi, I., Aizawa, S., Matsuyama, T., Mak, T. W., Taki, S., and Taniguchi, T. DNA damage-induced apoptosis and Ice gene induction in mitogenically activated T lymphocytes require IRF-1, Leukemia. 11 Suppl 3: 439-40, 1997.

27. Yamada, G., Ogawa, M., Akagi, K., Miyamoto, H., Nakano, N., Itoh, S., Miyazaki, J., Nishikawa, S., Yamamura, K., and Taniguchi, T. Specific depletion of the B-cell population induced by aberrant expression of human interferon regulatory factor 1 gene in transgenic mice, Proc Natl Acad Sci USA. 88: 532-6, 1991.

28. Willman, C. L., Sever, C. E., Pallavicini, M. G., Harada, H., Tanaka, N., Slovak, M. L., Yamamoto, H., Harada, K., Meeker, T. C., List, A. F., and et al. Deletion of IRF-1, mapping to chromosome 5q31.1, in human leukemia and preleukemic myelodysplasia, Science. 259: 968-71, 1993.

29. Tanaka, N., Ishihara, M., and Taniguchi, T. Suppression of c-myc or fosB-induced cell transformation by the transcription factor IRF-1, Cancer Lett. 83: 191-6, 1994.

30. Nozawa, H., Oda, E., Nakao, K., Ishihara, M., Ueda, S., Yokochi, T., Ogasawara, K., Nakatsuru, Y., Shimizu, S., Ohira, Y., Hioki, K., Aizawa, S., Ishikawa, T., Katsuki, M., Muto, T., Taniguchi, T., and Tanaka, N. Loss of transcription factor IRF-1 affects tumor susceptibility in mice carrying the Ha-ras transgene or nullizygosity for p53, Genes Dev. 13: 1240-5, 1999.

31. Hobart, M., Ramassar, V., Goes, N., Urmson, J., and Halloran, P. F. The induction of class I and II major histocompatibility complex by allogeneic stimulation is dependent on the transcription factor interferon regulatory factor 1 (IRF-1): observations in IRF-1 knockout mice, Transplantation. 62: 1895-901, 1996.

32. Salkowski, C. A., Barber, S. A., Detore, G. R., and Vogel, S. N. Differential dysregulation of nitric oxide production in macrophages with targeted disruptions in IFN regulatory factor-1 and -2 genes, J Immunol. 156: 3107-10, 1996.

33. Ogasawara, K., Hida, S., Azimi, N., Tagaya, Y., Sato, T., Yokochi-Fukuda, T., Waldmann, T. A., Taniguchi, T., and Taki, S. Requirement for IRF-1 in the microenvironment supporting development of natural killer cells, Nature. 391: 700-3, 1998.

34. Harada, H., Willison, K., Sakakibara, J., Miyamoto, M., Fujita, T., and Taniguchi, T. Absence of the type I IFN system in EC cells: transcriptional activator (IRF-1) and repressor (IRF-2) genes are developmentally regulated, Cell. 63: 303-12, 1990.

35. Kirchhoff, S., Wilhelm, D., Angel, P., and Hauser, H. NFkappaB activation is required for interferon regulatory factor-1-mediated interferon beta induction, Eur J Biochem. 261: 546-54, 1999.

36. Ohteki, T., Yoshida, H., Matsuyama, T., Duncan, G. S., Mak, T. W., and Ohashi, P. S. The transcription factor interferon regulatory factor 1 (IRF-1) is important during the maturation of natural killer 1.1+ T cell receptor-alpha/beta+ (NK1+ T) cells, natural killer cells, and intestinal intraepithelial T cells, J Exp Med. 187: 967-72, 1998.

37. Tanaka, N., Ishihara, M., Lamphier, M. S., Nozawa, H., Matsuyama, T., Mak, T. W., Aizawa, S., Tokino, T., Oren, M., and Taniguchi, T. Cooperation of the tumour suppressors IRF-1 and p53 in response to DNA damage, Nature. 382: 816-8, 1996.

38. White, L. C., Wright, K. L., Felix, N. J., Ruffner, H., Reis, L. F., Pine, R., and Ting, J. P. Regulation of LMP2 and TAP1 genes by IRF-1 explains the paucity of CD8+ T cells in IRF-1−/− mice, Immunity. 5: 365-76, 1996.

39. Foss, G. S. and Prydz, H. Interferon regulatory factor 1 mediates the interferon-gamma induction of the human immunoproteasome subunit multicatalytic endopeptidase complex-like 1, J Biol Chem. 274: 35196-202, 1999.

40. Yim, J. H., Wu, S. J., Casey, M. J., Norton, J. A., and Doherty, G. M. IFN regulatory factor-1 gene transfer into an aggressive, nonimmunogenic sarcoma suppresses the malignant phenotype and enhances immunogenicity in syngeneic mice, J Immunol. 158: 1284-92, 1997.

41. Di Bisceglie, A. M., Carithers Jr., R. L., and Gores, G. J. Hepatocellular carcinoma, Hepatology. 28: 1161-1165, 1998.

42. Vara, J. A., Portela, A., Ortin, J., and Jimenez, A. Expression in mammalian cells of a gene from Streptomyces alboniger conferring puromycin resistance, Nucleic Acids Res. 14: 4617-24, 1986.

43. Dinter, H. and Hauser, H. Superinduction of the human interferon-beta promoter, Embo J. 6: 599-604, 1987.

44. Grimm, C., Ortmann, D., Mohr, L., Michalak, S., Krohne, T. U., Meckel, S., Eisele, S., Encke, J., Blum, H. E., and Geissler, M. Treatment of hepatocellular carcinoma by breaking immunological ignorance towards alpha-fetoprotein using genetic immunization in mice, Gastroenterology 119: 1104-1112, 2000.

45. Schirmbeck, R., Wild, J., Blum, H. E., Chisari, F. V., Geissler, M., and Reimann, J. Ongoing T1 or T2 immune responses to the hepatitis B surface antigen (HBsAg) are excluded from the liver of mice in which transgene-encoded HBsAg is expressed, J. Immunol. 164: 4235-4243, 2000.

46. Wild, J., Grusby, M. J., Schirmbeck, R., and Reimann, J. Priming MHC-I-restricted cytotoxic T lymphocyte responses to exogenous hepatitis B surface antigen is CD4+ T cell dependent, J. Immunol. 163: 1880-1887, 1999.

47. Nicoletti, I., Migliorati, G., Pagliacci, M. C., Grignani, F., and Riccardi, C. A rapid and simple method for measuring thymocyte apoptosis by propidium iodide staining and flow cytometry, J Immunol Methods. 139: 271-9, 1991.

48. Fadok, V. A., Voelker, D. R., Campbell, P. A., Cohen, J. J., Bratton, D. L., and Henson, P. M. Exposure of phosphatidylserine on the surface of apoptotic lymphocytes triggers specific recognition and removal by macrophages, J Immunol. 148: 2207-16, 1992.

49. Tora, L., Mullick, A., Metzger, D., Ponglikitmongkol, M., Park, I., and Chambon, P. The cloned human oestrogen receptor contains a mutation which alters its hormone binding properties, Embo J. 8: 1981-6, 1989.

50. Danielian, P. S., White, R., Hoare, S. A., Fawell, S. E., and Parker, M. G. Identification of residues in the estrogen receptor that confer differential sensitivity to estrogen and hydroxytamoxifen, Mol Endocrinol. 7: 232-40, 1993.

51. Harada, H., Kitagawa, M., Tanaka, N., Yamamoto, H., Harada, K., Ishihara, M., and Taniguchi, T. Anti-oncogenic and oncogenic potentials of interferon regulatory factors-1 and -2, Science. 259: 971-4, 1993.

52. Futaki, M., Inokuchi, K., Hanawa, H., Tanosaki, S., Dan, K., and Nomura, T. Possible transforming activity of interferon regulatory factor 2 in tumorigenicity assay of NIH3T3 cells transfected with DNA from chronic myelogenous leukemia patients, Leuk Res. 20: 601-5, 1996.

53. Tanaka, H. and Samuel, C. E. Mechanism of interferon action: structure of the mouse PKR gene encoding the interferon-inducible RNA-dependent protein kinase, Proc Natl Acad Sci USA. 91: 7995-9, 1994.

54. Duncan, G. S., Mittrucker, H. W., Kagi, D., Matsuyama, T., and Mak, T. W. The transcription factor interferon regulatory factor-1 is essential for natural killer cell function in vivo, J Exp Med. 184: 2043-8, 1996.

55. Lohoff, M., Ferrick, D., Mittrucker, H. W., Duncan, G. S., Bischof, S., Rollinghoff, M., and Mak, T. W. Interferon regulatory factor-1 is required for a T helper 1 immune response in vivo, Immunity. 6: 681-9, 1997.

56. Drew, P. D., Franzoso, G., Becker, K. G., Bours, V., Carlson, L. M., Siebenlist, U., and Ozato, K. NF kappa B and interferon regulatory factor 1 physically interact and synergistically induce major histocompatibility class I gene expression, J Interferon Cytokine Res. 15: 1037-45, 1995.

57. Massa, P. T. and Wu, H. Interferon regulatory factor element and interferon regulatory factor 1 in the induction of major histocompatibility complex class I genes in neural cells, J Interferon Cytokine Res. 15: 799-810, 1995.

58. Chatterjee-Kishore, M., Kishore, R., Hicklin, D. J., Marincola, F. M., and Ferrone, S. Different requirements for signal transducer and activator of transcription 1alpha and interferon regulatory factor 1 in the regulation of low molecular mass polypeptide 2 and transporter associated with antigen processing 1 gene expression, J Biol Chem. 273: 16177-83, 1998.

59. Hung, K., Hayashi, R., Lafond-Walker, A., Lowenstein, C., Pardoll, D., and Levitsky, H. The central role of CD4(+) T cells in the antitumor immune response, J Exp Med. 188: 2357-68, 1998.

60. Zajac, A. J., Murali-Krishna, K., Blattman, J. N., and Ahmed, R. Therapeutic vaccination against chronic viral infection: the importance of cooperation between CD4+ and CD8+ T cells, Curr Opin Immunol. 10: 444-9, 1998.

61. Geissler, M., Gesien, A., Tokushige, K., and Wands, J. R. Enhancement of cellular and humoral immune responses to hepatitis C virus (HCV) core protein using DNA based vaccines augmented with cytokine expressing plasmids, J Immunol. 158: 1231-1237, 1997.

62. Geissler, M., Bruss, V., Michalak, S., Ortmann, D., Wands, J. R., and Blum, H. E. Intracellular retention of hepatitis B virus surface proteins reduces the immune response augmenting effects of IL-2 after cytokine genetic coimmunizations, J. Virol. 73: 4284-4292, 1999.

63. Vollmer, C. M., Jr., Eilber, F. C., Butterfield, L. H., Ribas, A., Dissette, V. B., Koh, A., Montejo, L. D., Lee, M. C., Andrews, K. J., McBride, W. H., Glaspy, J. A., and Economou, J. S. Alpha-fetoprotein-specific genetic immunotherapy for hepatocellular carcinoma, Cancer Res. 59: 3064-7, 1999.

64. Spitzer D., Hauser H. and Wirth D. Technical repot. Complement-protected amphotrophic retroviruses from murine packaging cells. Human Gene Therapy 10, 1893-1902 (1999)

65. Unsinger, J., Kröger, A., Hauser, H., and Wirth, D.: Retroviral vectors for transduction of an autoregulated bidirectional expression cassette. Molecular Therapy, 4, 484-489. (2001)

66. Unsinger J., Lindenmaier W., Hauser H. and Wirth D. LTR-flanked autoregulated expression cassettes for homogeneous and strictly controlled expression in adenoviral vectors. submitted 2002

67. Colbère-Garpin F., Horodniceanu F., Khourilsky P., and Garapin A. C. A new dominant hybrid selective arker for higher eukaryotic cells. J Mol. Biol. 150, 1-13. (1981).

68. Köster, M., Kirchhoff, S., Schaper, F. and Hauser, H.: Proliferation control of mammlian cells by the tumor suppressor IRF-1. Cytotechnology, 18, 67-75 (1995)

69. Kirchhoff, S., Köster, M., Wirth, M., Schaper, F., Gossen, M., Bujard, H. and Hauser, H.: Identification of mammalian cell clones exhibiting highly regulated expression from inducible promoters. TIG, 11, 219-220 (1995)

70. Kröger A. Evaluierung der tumorsuppressiven Eigenschaften des Interferon Regulatory factor-one (IRF-1). Dissertation, Technische Universität Braunschweig (1999)

What is claimed is:

1. A vaccine comprising (i) a gene construct or a polynucleotide encoding a member of the IRF family, wherein the expression of the IRF family member can be activated, and (ii) one or more genes encoding polypeptides selected from the group consisting of viral, bacterial, fungal, and parasitic peptides or one or more antigen-encoding genes derived from tumor cells, for the treatment, prevention, protective treatment and/or prophylactic immunization against tumoral, infectious and/or immune diseases.

2. The vaccine of claim 1, wherein the member of the IRF family is selected from the group consisting of wild-type IRF-1, synthetic IRF-1, immunologically active IRF-1 variants, wild-type members of the IRF family other than IRF-1, synthetic members of the IRF family other than IRF-1, immunologically active variants of a member of the IRF family other than IRF-1, and fusion proteins thereof.

3. The vaccine of claim 1 for the treatment of mammals.

4. The vaccine of claim 1 for the treatment of humans.

5. The vaccine of claim 1, wherein the gene construct encodes a fusion protein comprising a member of the IRF family as one domain of the fusion protein and a foreign protein as another domain of the fusion protein, wherein the activity of the fusion protein can be switched on and off by chemical or physical means.

6. The vaccine of claim 1, wherein the gene construct encodes an IRF-1/hER fusion protein, wherein the activity of the fusion protein can be regulated by compounds with estrogenic or anti-estrogene activity.

7. The vaccine of claim 1, wherein the gene construct or the polynucleotide is suitable for transfer into mammalian cells.

8. The vaccine of claim 7, wherein the gene construct or the polynucleotide comprises a viral vector.

9. The vaccine of claim 7, wherein the gene construct or the polynucleotide comprises an adenoviral vector.

10. The vaccine of claim 1, wherein the expression can be switched on by chemical activation or by physical activation.

11. The vaccine of claim 1, wherein the expression can be switched on thermally or by irradiation.

12. The vaccine of claim 1, wherein the expression can be switched on by means of a regulatable promoter.

13. The vaccine of claim 1, wherein the expression can be switched on or off by an external stimulus.

14. The vaccine of claim 1, wherein the expression can be switched on or off by a tetracycline.

15. The vaccine of claim 1, wherein the gene construct or polynucleotide and the antigen encoding gene are provided by means of separate vectors, a vector which provides all components, or as polycistronic expression units.

16. The vaccine of claim 15, wherein the gene construct or polynucleotide and the antigen encoding gene are provided as viral or bacterial carrier.

17. A human antigen presenting cell (APC), wherein the cell has been subjected to gene transfer ex vivo with a gene construct or a polynucleotide as defined in claim 1 (feature (i)) and with genes as defined in claim 1 (feature (ii)).

18. The human cell of claim 17, wherein the cell has been charged by physical transduction, chemical transduction, or viral transduction.

19. The human cell of claim 18, wherein the cell has been charged by electroporation.

20. The human cell of claim 17, wherein the cell is an autologous cell or an allogenic cell.

21. The human cell of claim 17, wherein the cell is a tumor cell.

22. The human cell of claim 21, wherein the cell is selected from the group consisting of carcinoma cells, sarcoma cells, and cells of tumors derived from the hematopoietic system.

23. The human cell of claim 22, wherein the cell is a hepatocellular carcinoma cell.

24. The human cell of claims 17 for the treatment, prevention, protective treatment and/or prophylactic immunization against tumoral, infectious and/or immune diseases.

25. The human cell of claim 17, wherein the active level of the member of the IRF family is higher than that of IRF-1 induced by interferon-gamma.

26. A method for the treatment, prevention, protective treatment and/or prophylactic immunization against tumoral, infectious and/or immune diseases comprising administering the vaccine of claim 1 to a mammal.

27. The method of claim 26, wherein the mammal is a human.

28. A method for the treatment, prevention, protective treatment and/or prophylactic immunization against tumoral, infectious and/or immune diseases comprising administering the human cell of claim 17 to a mammal.

29. The method of claim 28, wherein the mammal is a human.
